(19) United States
(12) Patent Application Publication
(10) Pub. No.: US 2025/0099746 A1
(43) Pub. Date: Mar. 27, 2025

(54) METHOD AND APPARATUS FOR NONINVASIVE INHIBITION OF DEEP VEIN THROMBOSIS

(71) Applicant: StimMed LLC, Buffalo, NY (US)

(72) Inventors: Richard W. DUCHARME, East Aurora, NY (US); Bryce BEAMER, Albion, NY (US); Kari L. ANASTASIA, Buffalo, NY (US); Michael J. MILLER, Depew, NY (US); Adam SPARKS, Lancaster, NY (US); Ariana B. ALLMAN, Eden, NY (US); Martin DIECK, Palo Alto, CA (US); Robert E. KAPLAN, Buffalo, NY (US); James J. CZYRNY, Amherst, NY (US)

(73) Assignee: StimMed LLC, Buffalo, NY (US)

(21) Appl. No.: 18/807,768

(22) Filed: Aug. 16, 2024

Related U.S. Application Data
(60) Provisional application No. 63/520,476, filed on Aug. 18, 2023.

(51) Int. Cl. A61N 1/04; A61N 1/36

(52) U.S. Cl. CPC A61N 1/0484; A61N 1/0488; A61N 1/36031; A61N 1/0452; A61N 1/36003; A61N 1/36021

(57) ABSTRACT

Disclosed herein is a garment configured to deliver electrical current from a remote power supply to electrically stimulate tissue of a foot of an individual. The garment can comprise a fabric and can be shaped to fit over the foot and a portion of a leg of the individual. The fabric can be configured to cause the garment body to conform to the foot when worn by the individual. The garment can further comprise a first electrode region integrated within the garment body and configured to engage a plantar region of the foot. The garment can further comprise a second electrode region integrated within the garment body and spaced from the first electrode region and configured to engage the plantar region of the foot.

### RELATED APPLICATION INFORMATION

This application is a non-provisional of U.S. Provisional Application No. 63/520,476, filed on Aug. 18, 2023, the entirety of which is incorporated by reference.

### TECHNICAL FIELD

The devices and methods disclosed herein relate to electrical stimulation of muscles for pain management and the prevention of thrombosis and include electrical stimulation of muscles of the foot or of other regions of the body.

### BACKGROUND OF THE INVENTION

Electrical stimulation of muscles and nerves by applying electrodes over the skin is currently used for enhancing blood circulation, reducing blood clots, and scrambling the pain signal that reaches the brain in order to manage pain.

Patients undergoing surgery, anesthesia, and extended periods of bed rest or other inactivity are often susceptible to a condition known as deep vein thrombosis (DVT). DVT is a clotting of venous blood in the lower extremities or pelvis. This clotting occurs due to the absence of muscular activity required to pump the venous blood in the lower extremities, local vascular injury, or a hypercoagulable state. The condition can be life-threatening if a blood clot migrates to the lung, resulting in pulmonary embolism (PE) or otherwise interfering with cardiovascular circulation. More generally, venous thromboembolic disease (VTED) is a cause of significant morbidity and mortality for individuals immobilized after orthopedic surgery due to neurologic disorders, even during prolonged travel, and a variety of other conditions.

Since 1954, it has been known that prolonged dependency stasis, a state imposed by airplane flights, automobile trips, and even attendance at the theater, may bring on thrombosis. In 1977, it was shown that trips as short as three to four hours could induce DVT and PE.

DVT and related conditions may be controlled or alleviated by assisting blood circulation (venous return) in the muscles. Current approaches to prophylaxis include mechanical compression using pneumatic compression devices, anticoagulation therapy, and electrical stimulation of the muscles. Pneumatic compression equipment is often too cumbersome for mobile patients or during prolonged travel. Anticoagulation therapy carries the risk of bleeding complications and must be started several days in advance to be effective. Electric stimulation has advantages over the other two methods in that it can be started at the time prophylaxis is needed and can be portable using DC current sources.

Electrical stimulation can be used for pain management. The most common form of electrical stimulation used for pain management is transcutaneous electrical nerve stimulation (TENS) therapy, which can provide short-term pain relief. Electrical nerve stimulation and electrothermal therapy are used to relieve pain associated with various conditions, including back pain. For example, intradiscal electrothermal therapy (IDET) is a treatment option for people with low back pain resulting from intervertebral disc problems. In TENS therapy for pain management, a small, battery-operated device delivers a low-voltage electrical current through the skin via electrodes placed near the source of pain. The electricity from the electrodes stimulates nerves in the affected area and sends signals to the brain that “scramble” normal pain perception. TENS is not painful and has proven to be an effective therapy to mask pain.

The following patents and publications disclose methods and devices using electrical stimulation for the inhibition of deep vein thrombosis: U.S. Pat. No. 6,615,080; US20170113037; U.S. Pat. No. 10,213,593B2; US20170333695; US20170333707; and US20190143098, where each of which is incorporated by reference. In addition to these devices, it is desirable for improvement of such devices to increase adoption by users and medical professionals. The methods and devices described herein include such improvements as well as other additional design features.

### SUMMARY OF THE INVENTION

Disclosed herein is a garment configured to deliver electrical current from a remote power supply to electrically stimulate tissue of a foot of an individual. The garment can comprise a fabric and can be shaped to fit over the foot and a portion of a leg of the individual. The fabric can be configured to cause the garment body to conform to the foot when worn by the individual. The garment can further comprise a first electrode region integrated within the garment body and configured to engage a plantar region of the foot. The garment can further comprise a second electrode region integrated within the garment body and spaced from the first electrode region and configured to engage the plantar region of the foot.

The garment can further comprise an electrical connector located on a portion of the garment body that engages a dorsal region of the foot or an anterior portion of the leg to reduce formation of adhesions or bruising by application of an external force. The electrical connector can be configured to electrically connect to the remote power supply so that the remote power supply can be positioned away from the garment body. The garment can further comprise an electrical conductor extending along a portion of the garment body and electrically coupling the electrical connector with the first electrode region and the second electrode region. The first electrode region, the second electrode region, and the electrical conductor can comprise elastic properties to stretch and contract upon movement of the foot when placed on or worn by the individual.

The garment body can include a removable portion configured to at least partially detach from the garment body to permit direct inspection of portions of the foot while the garment body is positioned on the foot. The removable portion can comprise a toe section of the garment body. The removable portion can be fully removable from the garment body. The removable portion can comprise an openable seam along the garment body. The first electrode region and the second electrode region can comprise a fabric coated with an electrically conductive material. The electrical conductor can comprise an elastic core having a conductive fabric positioned about an exterior of the elastic core. The electrical conductor can comprise an elastic core having one or more conductive filaments positioned about an exterior of the elastic core. At least a portion of the electrical connector can comprise a magnetic material to permit magnetic coupling of the portion of the remote power supply. The garment body can comprise at least one channel, and where the electrical conductor extends through at least one channel. An exterior of the first electrode region and an exterior of the second electrode region can comprise an insulated surface.

The garment can further comprise a controller containing the remote power supply, the controller including a flexible cord having a connector configured to couple to the electrical connector. The controller can be configured to be positioned away from the garment body and is configured to control electrical stimulation of the foot. The controller can be configured to monitor an impedance of tissue in contact with the first electrode region or the second electrode region to adjust electrical stimulation. The controller can be configured to monitor a capacitance of tissue in contact with the first electrode region or the second electrode region to adjust electrical stimulation. The controller can be configured to self-calibrate by stimulating in one or more pre-defined increments and measuring a resulting signal change until achieving a predetermined threshold.

The garment can further comprise a motion-detecting sensor, where the controller is configured to pause or modulate electrical stimulation when the motion-detecting sensor detects movement of the individual. The controller can be configured for wireless communication with an external electronic device. The garment can further comprise one or more sensors configured to measure a biological parameter of the individual. The biological parameter can be selected from a group consisting of a pulse rate, blood oxygen saturation, blood pressure, and temperature. At least the first electrode region or the second electrode region can comprise a bar tack to connect to the electrical conductor. The garment can further comprise at least one anti-slip surface on the garment body.

### BRIEF DESCRIPTION OF THE DRAWINGS

FIG. 1 illustrates a perspective view of a garment according to one variation of the present invention.

FIG. 2 illustrates a bottom perspective view of two garments being worn by a user.

FIG. 3 illustrates a perspective view of another variation of the garment having a tether connection to a battery pod.

FIG. 4 illustrates a side view of a patient on a platform and an operator managing a controller to control electrical connection of the garments.

FIG. 5 illustrates a perspective view of another variation of the garment having a consecutive line of electrical conduction.

FIG. 6 illustrates a perspective view of another variation of the garment having split lines of electrical conduction.

FIGS. 7A-C illustrate one variation of a connector for the garment comprising a spring contact deformation release mechanism.

FIGS. 8A-C illustrate one variation of a connector for the garment comprising linear magnetic pogo pins.

FIGS. 9A-C illustrate one variation of a connector for the garment comprising radial magnetic pogo pins.

FIGS. 10A-C illustrate one variation of a connector for the garment comprising a snap fitting.

FIGS. 11A-C illustrate one variation of a connector for the garment comprising a snap fitting with pogo pins.

FIGS. 12A-C illustrate one variation of a connector for the garment comprising a coaxial spring connector.

FIGS. 13A-B illustrate one variation of a connector for the garment comprising a barrel tether.

FIGS. 14A-C illustrate one variation of a connector for the garment comprising a multi-pin tether.

### DETAILED DESCRIPTION

Devices and methods described herein address existing challenges when using electrical stimulation devices. While the features described herein are discussed in relation to use on the fee to address venous thromboembolism, including deep venous thrombosis and pulmonary embolisms, variations of the devices and methods can also be applied for other indications and/or body parts.

The methods and devices reduce the difficulty of attaching a stimulator to the device once the device is on a patient's foot. They also allow ease of access to controls once connected to the device by patients and/or caregivers. The improvements also include ease of observing readings on the device and/or accompanying control units. Ease of changing the power supply or batteries.

In addition, the devices allow for improved accessibility and interface challenges from the existing products, where such improvements include, but are not limited to, location of placement, accessibility, display, button features, etc. The devices can streamline device preparation for the user and improve the ease of therapy application. The devices can improve comfort and aesthetics for the patient. The devices can provide known stimulation parameters from the existing systems. The device can improve upon and develop value-added design features appropriate for the new device (e.g., compatibility with shoes, compliance monitoring, etc.). In an additional variation, the stimulation parameters can use custom stimulation parameters that maximize the effect (e.g., maximizing ejection volume and rate of blood flow). In some cases, the stimulation parameters can optimize blood flow by pulsing the stimulation on/off at some frequency as opposed to sustaining. While such parameters can maximum clinical benefit, these parameters might also permit reduced power consumption and/or reduced battery size. In an additional variation, the stimulation parameters can include algorithms that direct stimulation specifically to certain areas such as pressure points reflexology zones, different layers/depths of tissues/energy patterns/intensities across those targets or other areas whether individually, simultaneously or in certain sequences where the overall stimulation is derived to induce or accentuate the effects. Other benefits can comprise reducing the risk of the user falling via additional stimulation of the foot.

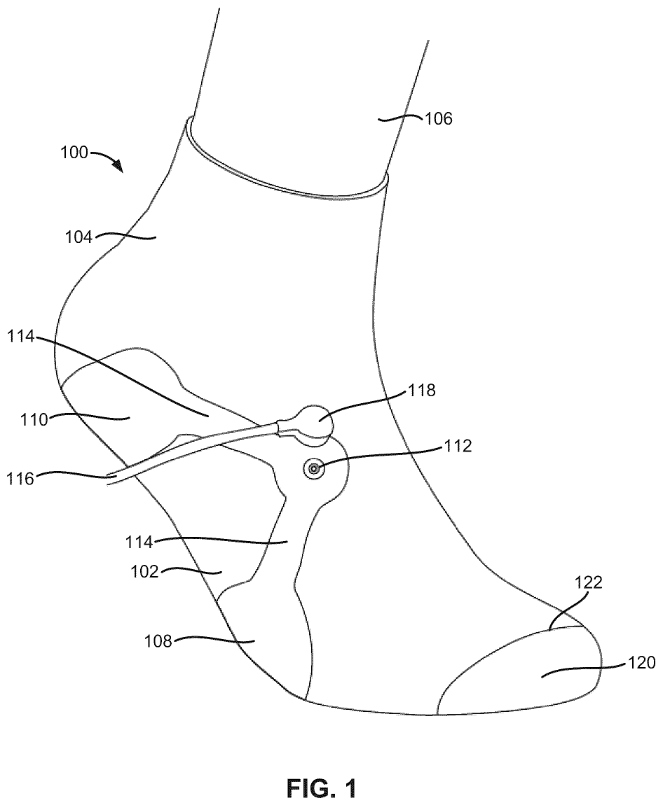

FIG. 1 illustrates a perspective view of a garment 100 according to one variation of the present invention. The garment 100 can comprise a foot portion 102 and an ankle portion 104. The garment 100 can comprise the shape of a sock and thus conform to the shape of a foot, though it should be understood that the concepts described in this invention can be used to treat various other parts of the body. The ankle portion 104 can cover part of a leg of the user 106.

The foot portion 102 can comprise one or more electrode regions 108, 110. The electrode regions 108 110 can comprise conductive electrodes within the body of the garment 100 and can engage a planar region of the foot. As seen in FIG. 1, the electrode regions 108 110 can comprise a substantially bulb-like shape, though various geometries and shapes can be used within the garment. The electrode regions 108 110 can be spaced apart from each other such that they contact spaced apart portions of the planar region of the foot. In other variations, the electrode regions 108 110 can be positioned at various regions on the garment 100.

The electrode regions 108 110 can comprise dry textile electrodes (e.g., silver coated nylon yarn coated) configured to deliver electrical current. This allows the electrode to be integrated directly into the garment 100 during manufacturing, eliminating the need for secondary processing of the garment 100. Alternatively, the electrodes can be made of stainless steel threads or carbon-infused yarn. In other variations of the garment 100, the electrode regions 108 110 can include either or both textile and/or gel electrode configurations. The exteriors of the electrode regions 108 110 can comprise insulated surfaces.

One technical problem encountered by the inventors is how to create electrodes that will not dry out from use, leading to frequent replacement for long-term wear. One technical solution determined by the inventors is to use dry textile electrodes, which hold advantages over other electrodes (e.g., gel electrodes), as the dry textile electrodes do not have the risk of drying out quickly and can be washed and reused. Additionally, such dry textile electrodes obviate the need for gels and adhesives, which can result in the garments being difficult to maintain.

An electrical connector 112 can be located on a portion of the garment 100. The electrical connector 112 can engage a dorsal region of the foot or an anterior or lateral portion of the leg to reduce the formation of adhesions, pressure ulcers, or bruising by application of an external force. The electrical connector 112 can be configured to electrically connect to the remote power supply such that the remote power supply can be positioned away from the garment body. In some variations, the electrical connector 112 can be configured to be tethered to a power supply via a cable (not shown in FIG. 1). In some variations, the electrical connector 112 can be configured to be directly connected to a battery pod.

The electrical connector 112 can be positioned on a portion of the foot that will not encounter force when a patient is lying in a bed. This can reduce the formation of adhesions or bruises from the electrical connector 112 against tissue.

The garment 100 can further comprise one or more electrical conductors 114 extending along a portion of the garment 100. The one or more electrical conductors 114 can electrically couple the electrical connector 112 with the first electrode region 108 and the second electrode region 110. The first electrode region 108, the second electrode region 110, and the electrical conductor 114 can each comprise elastic properties to stretch and contract upon movement of the foot when placed on or worn by the individual. The conductors 114 can transmit stimulation from the electrical connector 112 area to the electrode regions 108, 110.

A flexible stimulation cord 116 can be provided along with a magnetic portion 118 at one end to connect to the electrical connector 112. When connected, the stimulation cord 116 can provide power to the electrical connector 112 via a power supply tethered thereto. The electrical connector 112 can comprise a magnetic material such that the magnetic portion 118 of the stimulation cord 116 can couple thereto. The coupling of the magnetic portion 118 and the electrical connector 112 can comprise a 360-degree rotation for adjusting the patient as needed.

Stimulation of the intrinsic plantar foot muscles can cause these muscles to contract, compressing the plantar venous plexus. A wide range of stimulation patterns can be used to improve the blood flow in target areas. For example, a rotation of different stimulation rates within a defined time period can result in a combination of high blood flow velocity and high volumetric flow. This in turn can reduce the power consumption and battery size of the garment by shortening the duration of activation with a higher duty cycle, improving the battery life of the device.

The conductors 114 can comprise an elastic core and conductive yarn filaments braided or coiled around the elastic core. The elastic core can be made of thermoplastic polyurethane (TPU). In other variations, the elastic core can comprise thermoplastic elastomers, polyethylene, polypropylene, silicone rubber, polyvinyl chloride, or combinations thereof. The elastic core can comprise a durometer of about 90 A durometer. The elasticity of the cord can provide for stretch and relaxation of the garment 100 during use as well as comfort for the user 106 (i.e., the user does not feel the conductive yarn).

The first electrode region 108 and the second electrode region 110 can each comprise a bar tack to connect to the adjacent electrical conductor 114. In other variations, the first electrode region 108 and the second electrode region 110 can each connect to the adjacent electrical conductor 114 via thermoplastic polyurethane (TPU) adhesive lamination.

This configuration can provide for seamless integration into garment 100 through a knit channel within the garment 100. Accordingly, the integration of the conductor does not modify the overall profile of the garment 100, allowing the garment 100 to be easily worn with a shoe. This results in the garment 100 comprising flexibility and stretchability while maintaining electrical conductance throughout the garment 100.

The conductive yarn can be helically wrapped around an exterior of the elastic core. To insulate the conductor 114, a non-conductive textile can be braided or coiled around the conductive textile to create an insulated conductor 114. The number/ratio of conductive to non-conductive fibers can be varied to tailor conductivity. In other variations, the conductor 114 can be insulated by reflow processes, lamination covers, or heat-shrinking.

In other variations, the conductor 114 can comprise a silver-plated fabric trace that assists in transmitting stimulation from the electrical connector 112 to the electrode regions 108, 110. The silver-plated fabric can be insulated by a laminate cover. The silver-plated fabric can stretch with the user's foot as the foot changes position during use of the garment 100. To terminate the conductor 114, the silver-plated fabric can be sewn at the electrode regions 108, 110 using conductive thread.

The conductor 114 can be sewn into the garment 100 such that the conductive yarn or silver-plated fabric trace on the elastic core is exposed and sewn to the conductive electrode regions 108, 110 on the garment 100. The conductor 114 can be sewn using conductive yarn or thread to maintain continuity of conductivity and can be sewn using standard garment sewing techniques. Further, this termination method does not require additional connectors to integrate the conductors 114 into the garment 100.

In other variations, the termination of the elastic core can comprise one or more conductive textile yarns braided or wrapped along the elastic core. The resulting assembly can be protected by a non-conductive braided or elastomeric sheath. Ultrasonic or heat welding can be used to fuse the ends of the core and the sheath. Conductive sewing threads can be used to tack the elastic core in place and to offer additional mechanical strain relief. These threads can also provide a contact-based connection between the conductive textile yarns and the electrode regions 108, 110 of the garment 100.

In other variations, the elastic core can be folded in half to create a loop at termination.

In other variations, the elastic core can be instead provided with a silver-coated nylon fabric with elastane content at termination. The fabric can serve as both the elastic core and the conductive member and can be flat and sewn onto the garment 100.

The garment 100 can further comprise a removable portion 120 connected to the remainder of the garment 100 via an openable seam 122. The removable portion 120 can be used to expose the toes of the user, which can allow for visual inspection of a patient's toes or other portions of the feet and monitoring of circulation and/or nerve function. The removable portion 120 can maintain attachment to the garment 100 (i.e., partially detached) or can be fully removed from the garment 100 itself.

In other variations, the conductors 114 can be used for other applications or devices that experience high stress and/or vibratory environments. Such examples include industrial applications, automotive applications, and the like. The flexibility of the conductors 114 can be beneficial in such environments to reduce transmission of vibration. In other variations, the conductors 114 can be used in various other items of clothing.

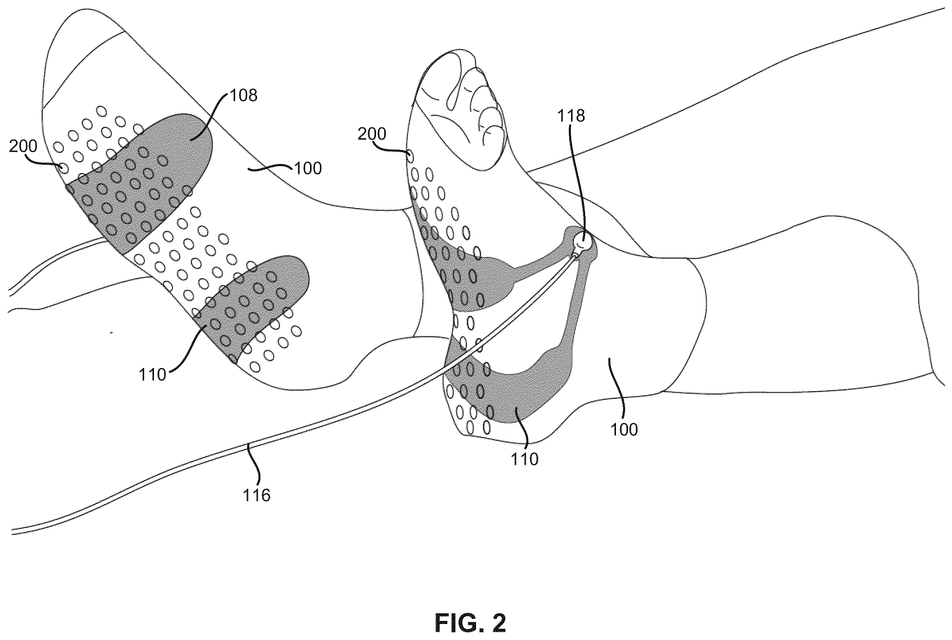

FIG. 2 illustrates a bottom perspective view of two garments being worn by a user. This view illustrates the magnetic portion 118 coupled to the electrical connector 112 for stimulation of the feet. The removable portion 120 (FIG. 1) of the garment 100 has been removed for visual inspection of the toes or toe section of the user.

The garment 100 can further comprise one or more anti-slip features 200 that provide added traction for the user when walking. The anti-slip features 200 can be made of a silicone pattern and can be spaced apart on the plantar surface of the garment 100.

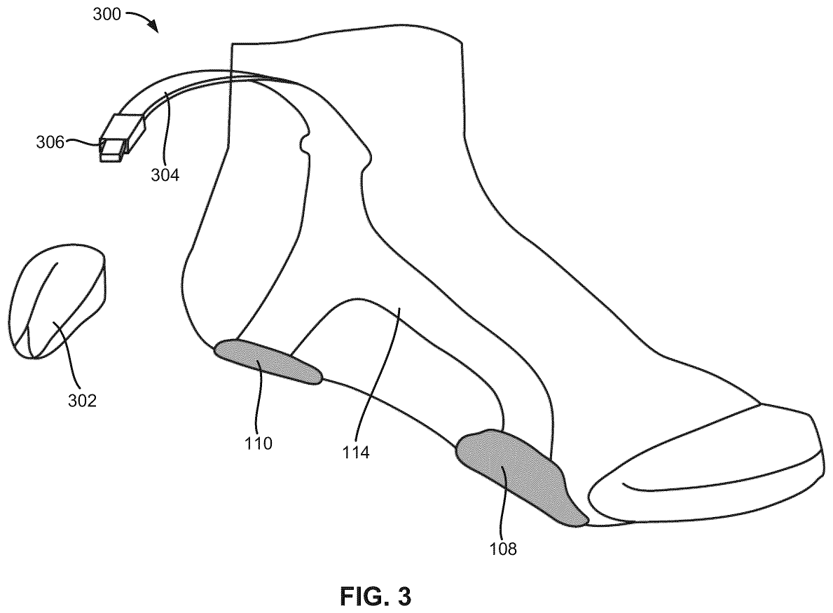

FIG. 3 illustrates a perspective view of another variation of a garment 300 having a tether connection to a battery pod 302. The battery pod 302 can comprise an internal stimulator and can connect to a tether 304, which connects to the electrode regions 108, 110 via a conductor 114. The battery pod 302 can be removable from a tether connector 306 of the tether 304. The battery pod 302 can comprise a clip feature to couple to the garment 100 or a shoe if the user desires to walk. The tether 304 can comprise an electrical connector 112 to provide stimulation to the garment 100 accordingly.

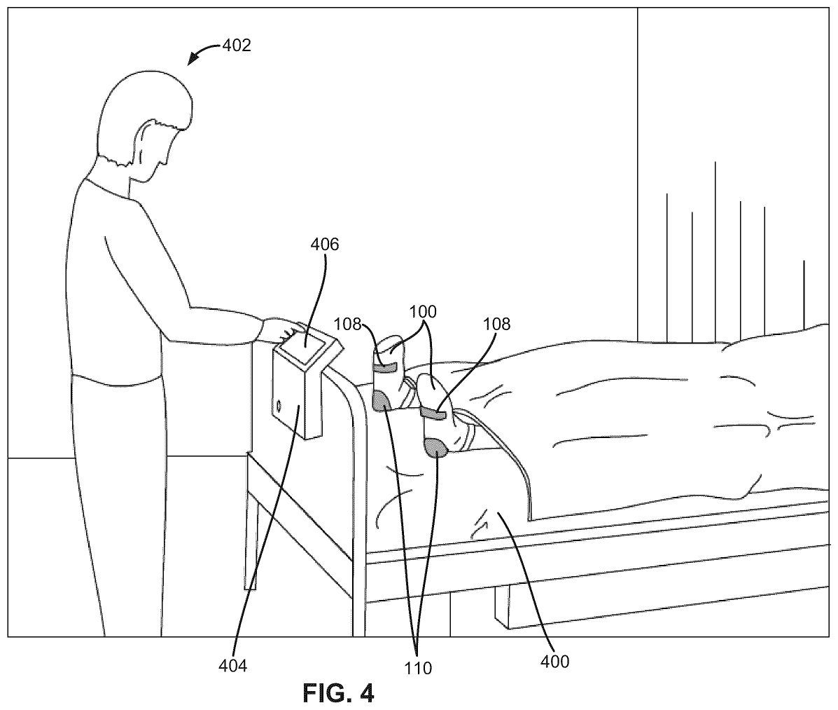

FIG. 4 illustrates a side view of a patient on a platform 400 and an operator 402 managing a system having a controller 404 to control the electrical connection of the garments 100. The controller 404 can be a bedside controller that is in wireless communication with the stimulator in the battery pod 302 connected to a tether of the garment 100 (see FIG. 3 above) in order to stimulate the electrical components of the garment 100. In other variations, the controller 404 can be in a tethered connection with the electrical connector 112 of the garment 100. In other variations, the controller 404 can be in remote communication with the electrical connector 112 of the garment 100.

In other variations, the controller 404 can be configured for wireless communication with an external electronic device (e.g., a cellular phone). Accordingly, the controller 404 can comprise a remote power supply.

The controller 404 can comprise a display 406 with a user interface for the operator's control. The display 406 can comprise a screen of current settings (e.g., stimulation duration, intensity), a display of the duration of the current treatment, an indicator for stimulation to turn either or both garments 100 on or off, and a battery status indicator.

In other variations, the controller 404 can comprise a flexible cord having a connector configured to couple to the electrical connector 112.

In variations that comprise a remote connection, the garment 100 can comprise an electrical connector 112 to provide for a wired connection via the stimulation cord 116 if desired.

The controller 404 can also be provided with systems for real-time automatic stimulation intensity adjustment capabilities. For example, the impedance and/or capacitance of the garment 100 can be monitored live via one or more sensors to adjust stimulation levels of the electrode regions 108, 110 accordingly. In other variations, a voltage change and/or rise and decay rate can be monitored live to adjust stimulation levels of the electrode regions 108, 110 accordingly.

In other variations, the controller 404 can self-calibrate by stimulating the electrode regions 108, 110 in pre-defined increments. Accordingly, the controller 404 can measure resulting signal changes until a predetermined threshold is achieved.

In other variations, the controller 404 can also be provided with motion sensing capabilities (e.g., an accelerometer or other motion-detecting sensors) to pause or modulate stimulation upon detection of movement of the patient and/or to establish a minimum stimulation level that is individualized for the user.

In other variations, the controller 404 can provide for stimulation feedback based on muscle twitch/toe curl via accelerometer within the garment 100.

In other variations, the controller 404 can increase stimulation intensity until the detection of a muscle contraction or toe curl via an accelerometer. The controller 404 can then modulate the electrical signal.

In other variations, the controller 404 can be configured to automatically shut off stimulation when it detects the user walking. In other variations, a force sensor can be provided and triggered when a user is standing.

In other variations, the garment 100 can comprise integrated sensors that can monitor data for monitoring biological parameters such as patient vitals, including pulse rate, pulse oximetry, respiration rate, blood pressure, temperature, blood oxygen saturation, and patient movement. Such data can be logged and saved to share with relevant parties (e.g., clinicians, insurance companies, etc.) for care or compliance purposes. The data can further comprise the duration of therapy and the stimulation intensity at relevant intervals during therapy.

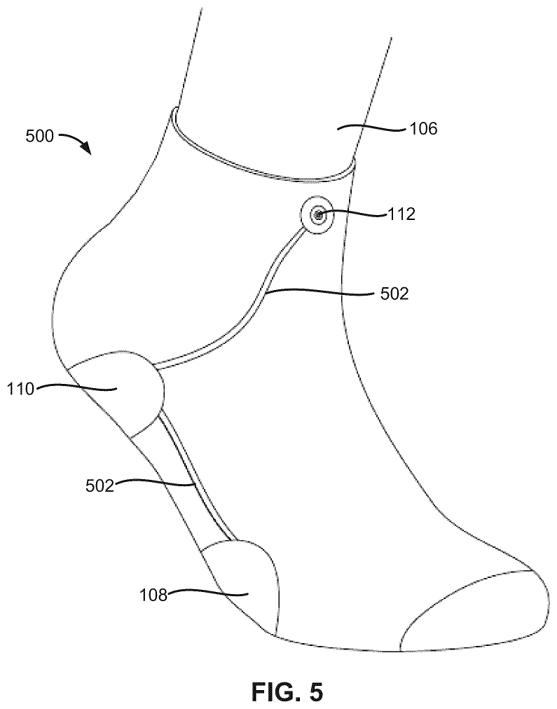

FIG. 5 illustrates a perspective view of another variation of a garment 500 having a consecutive line of electrical conduction. In this variation, the conductor 502 can comprise a single line connecting the electrode regions 108, 110 and the electrical connector 112.

The electrical connector 112 can be positioned at the top of the garment 100 near the ankle and can comprise a magnetic component to connect with a stimulation cord 116.

In other variations, a battery pod 302 can be used to connect to the electrical connector 112. The battery pod 302 can comprise a controller within and can be controlled via a wireless external device (e.g., via a cellular phone). The application can comprise a display 406 with a user interface for the operator's control. The display 406 can comprise a display of current settings (e.g., stimulation duration, intensity), a display of the duration of the current treatment, an indicator for stimulation to turn either or both garments 100 on or off, and a battery status indicator. As discussed with reference to FIG. 3, the battery pod 302 can be movable or stationary.

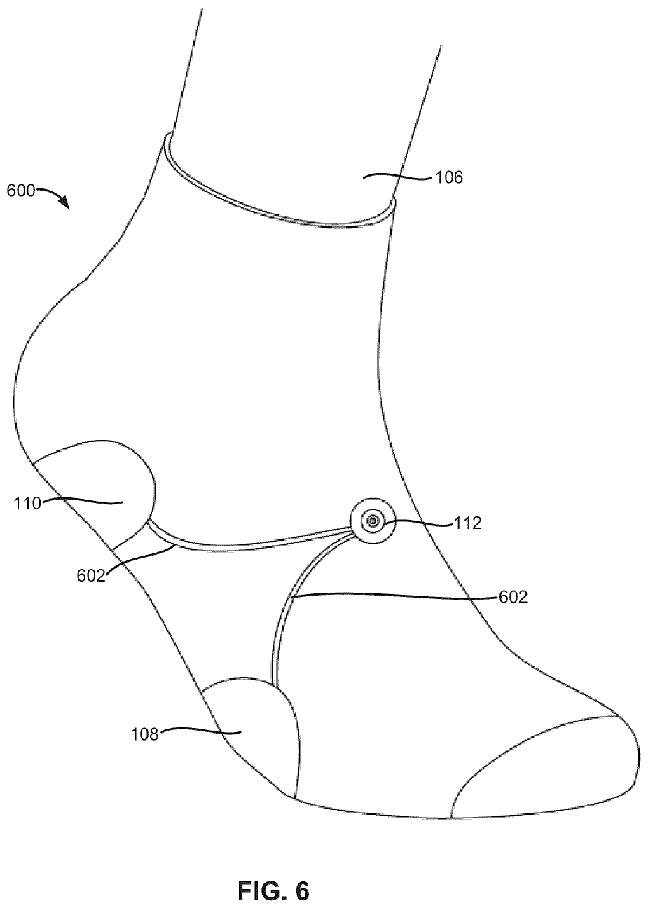

FIG. 6 illustrates a perspective view of another variation of a garment 600 having split lines of electrical conduction. In this variation, the conductor 602 can be a braided cable that runs through the garment 100 between each separate electrode region 108, 110 and the electrical connector 112.

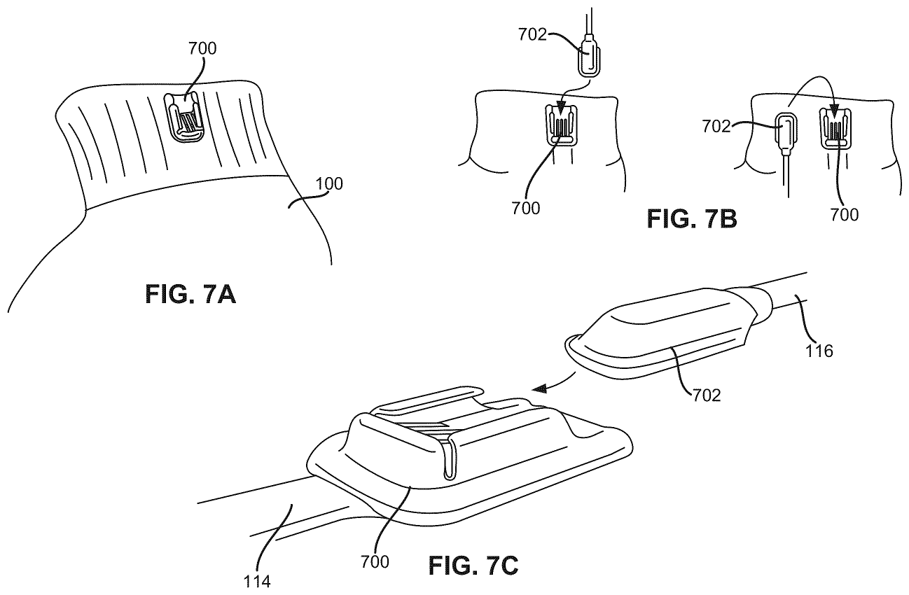

FIGS. 7A to 14C illustrate variations for connecting the garment to the stimulation cord 116 that connects to the main stimulation module or battery.

FIGS. 7A-C illustrate one variation of a connector for the garment comprising a spring contact deformation release mechanism. A receptacle 700 can be placed at the top of the garment 100 near the ankle of the user. The receptacle 700 can be substantially rectangular and can comprise a detent for receiving a cord head 702 of the stimulation cord. This locks the cord head 702 into the receptacle 700. When ready to be released, out-of-plane forces can cause deformation of the elastomeric receptacle 700, causing release of the connection. In this variation, three receptacle pads can be used for polarity and reversibility, though it should be understood that any number of receptacle pads can be used.

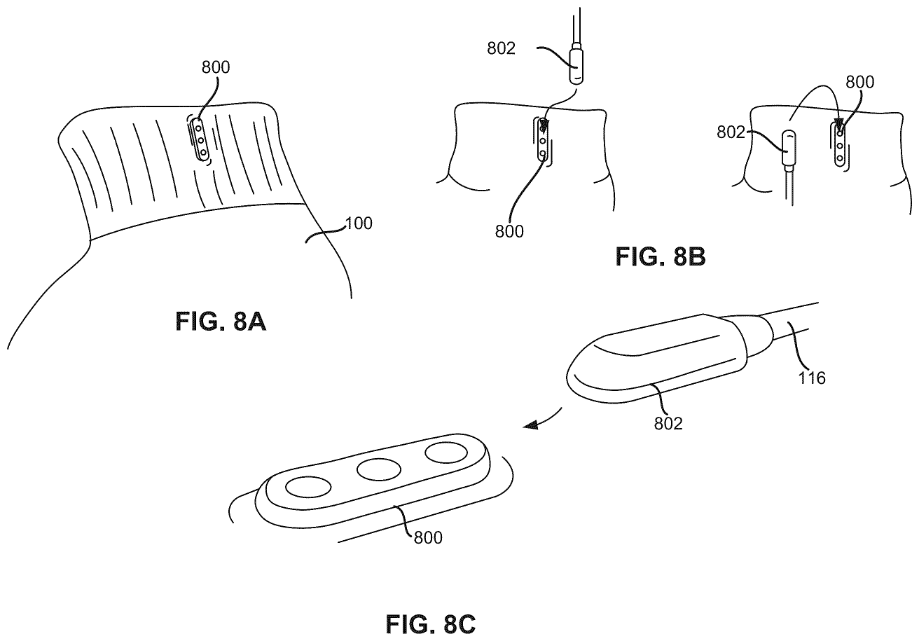

FIGS. 8A-C illustrate one variation of a connector for the garment comprising linear magnetic pogo pins. A landing pad 800 can be placed at the top of the garment 100 near the ankle of the user. The landing pad 800 can couple to one or more pogo pins on the cord head 802 for the electrical connection. The landing pad 800 and the cord head 802 can comprise magnets to provide for the necessary contact therebetween and to allow for strain release when necessary. In this variation, three individual pads can be used for polarity and reversibility, though it should be understood that any number of receptacle pads can be used.

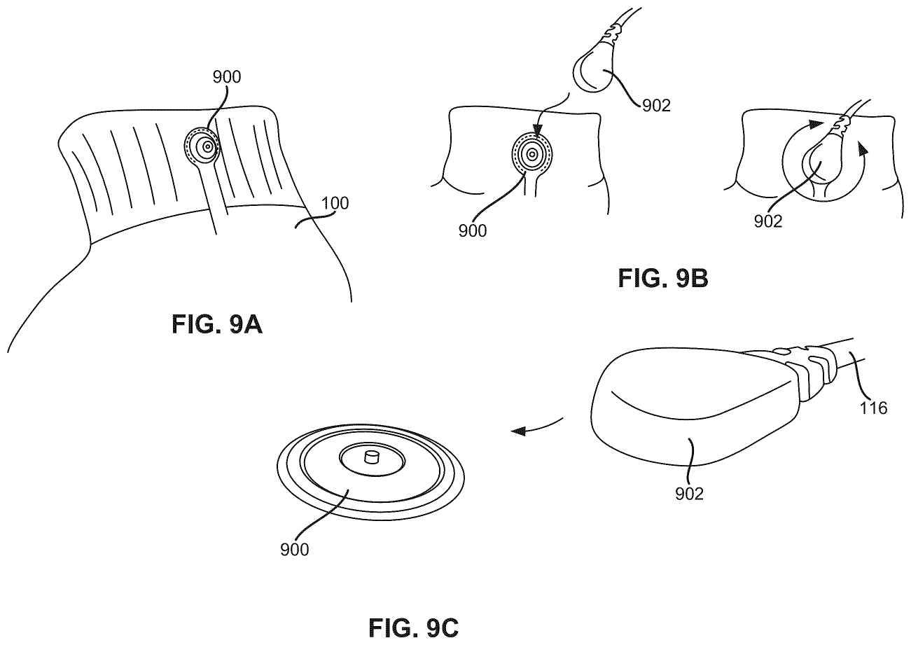

FIGS. 9A-C illustrate one variation of a connector for the garment comprising radial magnetic pogo pins. A radial hub 900 can be placed at the top of the garment 100 near the ankle of the user. The radial hub 900 can couple to one or more pogo pins on the radial connector 902 at the end of the stimulation cord 116. The radial hub 900 and the radial connector 902 can comprise a magnetic coupling therebetween to depress the pogo pin connections at the center of the radial connector 902. The magnet itself can comprise the ground connection. The radial nature of this connection allows the radial connector 902 to rotate freely and ensure correct polarity.

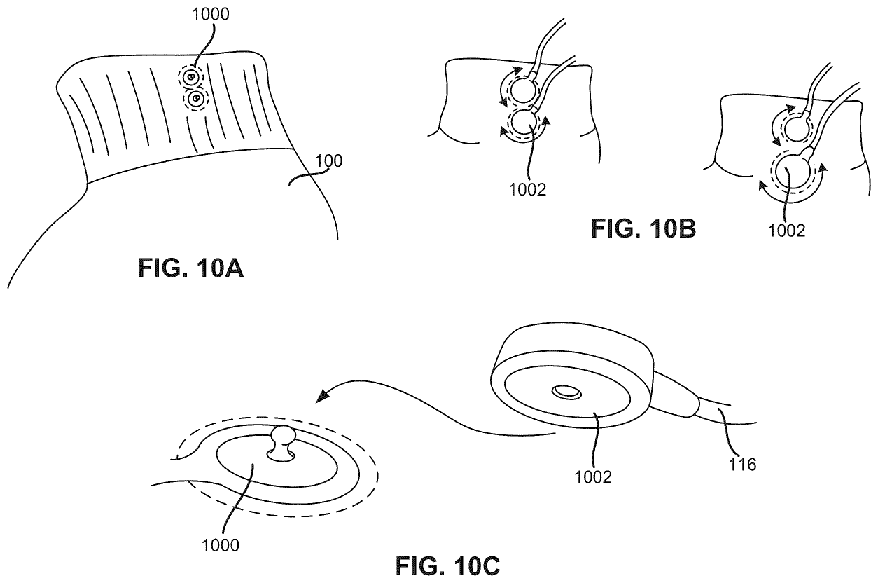

FIGS. 10A-C illustrate one variation of a connector for the garment comprising a snap fitting. One or more snap fittings 1000 can be placed at the top of the garment 100 near the ankle of the user. The snap fittings 1000 can be stainless steel and can provide electrical contacts at their respective locations. The snap fittings 1000 can independently rotate and can offer a strain release via their connection to the snap connector 1002 at the end of the stimulation cord 116. The geometry of the snap fittings 1000 can be made unique to ensure correct polarity.

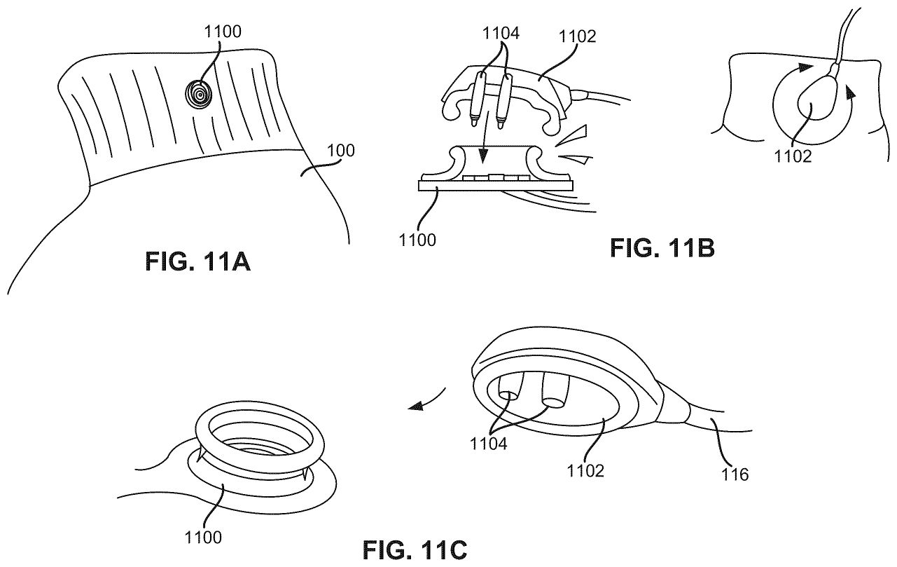

FIGS. 11A-C illustrate one variation of a connector for the garment comprising a snap fitting with pogo pins. A radial snap receptacle 1100 can be placed at the top of the garment 100 near the ankle of the user and can be made of stainless steel or plastic. The radial snap receptacle 1100 can be a mechanical snap with embedded PCB/pogo pin connectors therein. The radial snap receptacle 1100 can click onto a radial snap connector 1102 at the end of the stimulation cord 116. The radial snap connector 1102 can comprise one or more pogo pins 1104 that couple with the radial snap receptacle 1100. The offset pogo configuration can result in a matched polarity and can provide dynamic rotation of the connection.

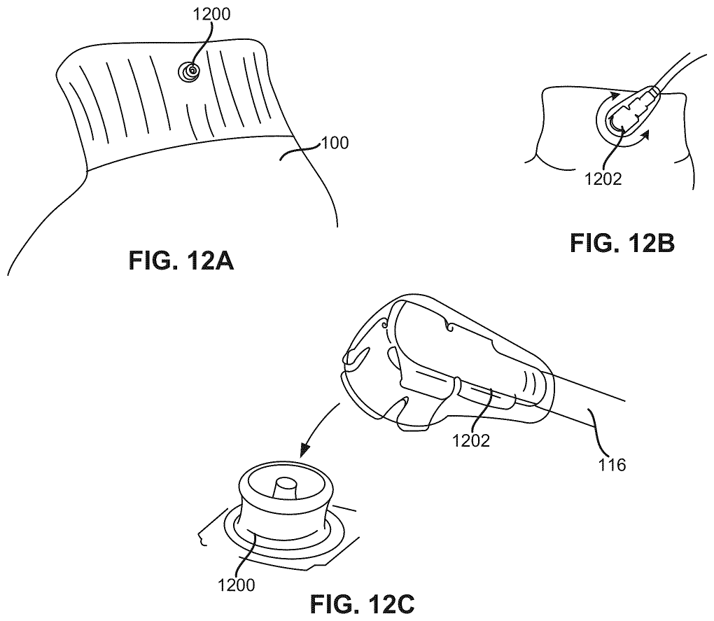

FIGS. 12A-C illustrate one variation of a connector for the garment comprising a coaxial spring connector 1200. The spring connector 1200 can be placed at the top of the garment 100 near the ankle of the user. The spring connector 1200 can be coupled to a spring connector head 1202 that can rotate when coupled with the spring connector 1200. The coupling can comprise a strain release.

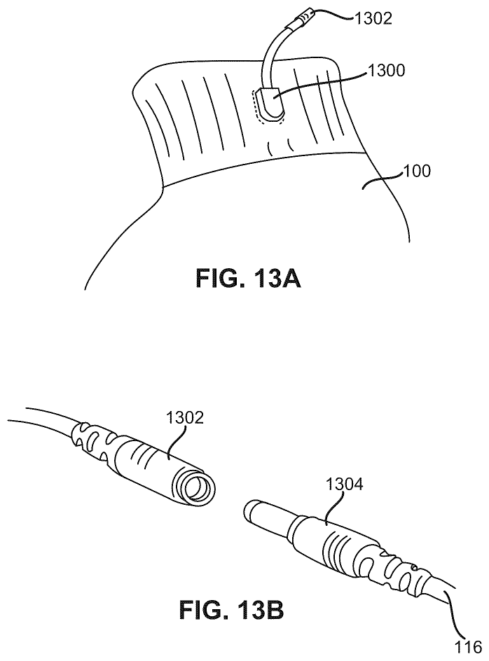

FIGS. 13A-B illustrate one variation of a connector for the garment comprising a barrel tether. A barrel receptacle 1300 can be placed at the top of the garment 100 near the ankle of the user. The barrel receptacle 1300 can comprise a barrel connector 1302 which can couple to a tether connector 1304 of the stimulation cord 116. Short tethers from the garment 100 can allow the barrel connector 1302 to align with the direction of strain to allow the release of the barrel connector 1302.

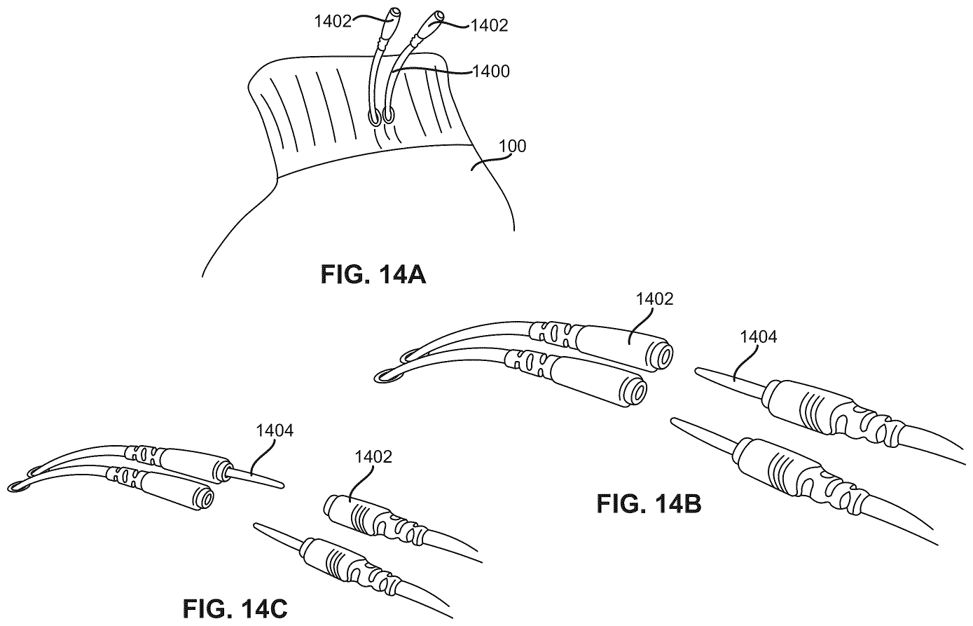

FIGS. 14A-C illustrate one variation of a connector for the garment comprising a multi-pin tether. One or more tethers 1400 comprising receptacles 1402 extending from the garment 100 can align with the direction of strain to allow release of the one or more pins 1404, which can couple onto the receptacles. FIG. 14C illustrates another variation in which the garment 100 comprises two tethers, one extending a pin 1404 and the other extending a receptacle 1402.

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

1. A garment configured to deliver electrical current from a remote power supply to electrically stimulate tissue of a foot of an individual, the garment comprising:
a garment body comprising a fabric and shaped to fit over the foot and a portion of a leg of the individual, wherein the fabric is configured to cause the garment body to conform to the foot when worn by the individual;
a first electrode region integrated within the garment body and configured to engage a plantar region of the foot;
a second electrode region integrated within the garment body and spaced from the first electrode region and configured to engage the plantar region of the foot;
an electrical connector located on a portion of the garment body that engages a dorsal region of the foot or an anterior portion of the leg to reduce formation of adhesions or bruising by application of an external force, the electrical connector configured to electrically connect to the remote power supply such that the remote power supply can be positioned away from the garment body; and
an electrical conductor extending along a portion of the garment body and electrically coupling the electrical connector with the first electrode region and the second electrode region;
wherein the first electrode region, the second electrode region, and the electrical conductor comprise elastic properties to stretch and contract upon movement of the foot when placed on or worn by the individual.

2. The garment of claim 1, wherein the garment body includes a removable portion configured to at least partially detach from the garment body to permit direct inspection of portions of the foot while the garment body is positioned on the foot.

3. The garment of claim 2, where the removable portion comprises a toe section of the garment body.

4. The garment of claim 2, wherein the removable portion is fully removable from the garment body.

5. The garment of claim 2, wherein the removable portion comprises an openable seam along the garment body.

6. The garment of claim 1, wherein the first electrode region and the second electrode region comprise a fabric coated with an electrically conductive material.

7. The garment of claim 1, wherein the electrical conductor comprises an elastic core having a conductive fabric positioned about an exterior of the elastic core.

8. The garment of claim 1, wherein the electrical conductor comprises an elastic core having one or more conductive filaments positioned about an exterior of the elastic core.

9. The garment of claim 1, wherein at least a portion of the electrical connector comprises a magnetic material to permit magnetic coupling of the portion of the remote power supply.

10. The garment of claim 1, wherein the garment body comprises at least one channel and where the electrical conductor extends through at least one channel.

11. The garment of claim 1, where an exterior of the first electrode region and an exterior of the second electrode region comprise an insulated surface.

12. The garment of claim 1, further comprising a controller containing the remote power supply, the controller including a flexible cord having a connector configured to couple to the electrical connector, wherein the controller is configured to be positioned away from the garment body and is configured to control electrical stimulation of the foot.

13. The garment of claim 12, wherein the controller is configured to monitor an impedance of tissue in contact with the first electrode region or the second electrode region to adjust electrical stimulation.

14. The garment of claim 12, wherein the controller is configured to monitor a capacitance of tissue in contact with the first electrode region or the second electrode region to adjust electrical stimulation.

15. The garment of claim 12, wherein the controller is configured to self-calibrate by stimulating in one or more pre-defined increments and measuring a resulting signal change until achieving a predetermined threshold.

16. The garment of claim 12, further comprising a motion detecting sensor, where the controller is configured to pause or modulate electrical stimulation when the motion detecting sensor detects movement of the individual.

17. The garment of claim 12, wherein the controller is configured for wireless communication with an external electronic device.

18. The garment of claim 1, further comprising one or more sensors configured to measure a biological parameter of the individual.

19. The garment of claim 18, wherein the biological parameter is selected from a group consisting of a pulse rate, blood oxygen saturation, blood pressure, and temperature.

20. The garment of claim 1, wherein at least the first electrode region or the second electrode region comprises a bar tack to connect to the electrical conductor.

21. The garment of claim 1, further comprising at least one anti-slip surface on the garment body.
